(19) United States
(12) Patent Application Publication
(10) Pub. No.: US 2025/0170197 A1
(43) Pub. Date: May 29, 2025

(54) CANNABIS EXTRACT COMPOSITIONS AND METHODS FOR TOPICAL DELIVERY FOR SKIN CARE

(71) Applicant: Gregory P. Pilant, Gordonsville, TN (US)

(72) Inventor: Gregory P. Pilant, Gordonsville, TN (US)

(21) Appl. No.: 18/960,416

(22) Filed: Nov. 26, 2024

Related U.S. Application Data
(60) Provisional application No. 63/602,951, filed on Nov. 27, 2023.

(51) Int. Cl. A61K 36/185; A61K 8/19; A61K 8/27; A61K 8/67; A61K 8/9789; A61K 9/00; A61K 31/00; A61K 31/519; A61K 31/555; A61K 33/00; A61K 33/06; A61K 33/30; A61Q 19/00

(52) U.S. Cl. CPC A61K 36/3482; A61K 8/19; A61K 8/27; A61K 8/673; A61K 8/9789; A61K 9/0014; A61K 31/519; A61K 31/555; A61K 31/658; A61K 33/00; A61K 33/06; A61K 33/30; A61Q 19/00; A61K 2800/10

(57) ABSTRACT

The present invention provides a topical composition for application to a keratinous tissue comprising a cosmetically or pharmaceutically acceptable carrier; one or more selected from potassium, zinc, calcium, rubidium disposed in the cosmetically or pharmaceutically acceptable carrier; and one or more cannabis extracts disposed in the cosmetically or pharmaceutically acceptable carrier.

### CROSS-REFERENCE TO RELATED APPLICATIONS

The present application claims the benefit under 35 U.S.C. 119 to U.S. Provisional Patent Application Ser. No. 63/602,951 filed on Nov. 27, 2023, the content of which is incorporated herein by reference in its entirety.

### TECHNICAL FIELD OF THE INVENTION

The present invention relates generally to methods and compositions for topical application which comprise one or more metals and a cannabis extract complex and therapeutically effective amount of a cannabis extract to provide specific benefits to the skin.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with topical application. Generally, consumers seek to improve the appearance of their skin and hair including the visible signs of aging, discoloration, hyper-pigmentation, redness, inflammation, and over-production of oils and/or lipids at the skin surface. In addition, collagen synthesis and degradation also play a role in common skin concerns. Collagen is the body's major structural protein and gives skin strength, durability, and a smooth, plump appearance. It is created by fibroblasts, specialized skin cells located in the dermis, in a process that involves conversion of preprocollagen I to procollagen I and eventually to tropocollagen, the form that forms collagen fibers. For example, a reduction in collagen I is associated with loss of firmness and elasticity of skin and leads to wrinkling associated with aging. Hyaluronic acid is another component of skin that plays a role in its aesthetic appearance including, for example, changes in tissue hydration, as well as plumpness and protection against free radicals. With age, however, glycosaminoglycan (GAG) synthesis and overall GAG skin content appear to decline.

U.S. Pat. No. 7,569,558, entitled, “Topical Delivery of Trace Metals for Skin Care,” discloses a method for topical delivery of trace metals for the modulation of certain metalloenzymes. The method of topical delivery of the present invention comprises; (i) mixing of a trace metal salt of a phosphorylated nitrogen heterocyclic base complexed with a chelating agent, and (ii) a carrier, and (iii) topical application of said mixture. The modulation of metalloenzymes such as Superoxide Dismutase, Elastase, Tyrosinase, Matrix metalloproteases, and Ubiquitin-Proteasome pathway by the methods of the present invention is useful for providing anti-inflammatory, skin whitening, wrinkles reduction, skin aging control, cellular antioxidant, acne control, hair growth modulation, and skin damage control benefits.

U.S. Pat. No. 8,575,106, entitled, “Cosmetic Uses of Modified Stressed Yeast Extracts and Related Compositions,” discloses cosmetic compositions comprising a metal-complexed peptide fraction of stressed yeast extracts and/or a calcium influx inhibitor are disclosed, as well as methods of using such compositions to impart exfoliating, anti-aging, anti-lipid, anti-inflammatory, and/or lightening benefits to the skin; and/or lightening benefits to the hair. These compositions are believed to have modulatory activity against at least one biochemical pathway implicated in skin aging, inflammation, lipid synthesis, and melanin production.

U.S. Patent Application Publication No. 20190307719, entitled, “Cannabis Composition,” discloses a method for treating a skin disorder. In particular, the invention provides a method for treating a skin disorder, comprising administering to a patient in need thereof an effective amount of the pharmaceutical composition comprising a Cannabis extract and optionally one or more pharmaceutically acceptable carriers, diluents, adjuvants, excipients or any combination thereof, the Cannabis extract comprising at least 75% by weight of a main cannabinoid.

### SUMMARY OF THE INVENTION

The present invention provides a topical composition for application to a keratinous tissue comprising: a cosmetically or pharmaceutically acceptable carrier; one or more selected from potassium, zinc, calcium, rubidium disposed in the cosmetically or pharmaceutically acceptable carrier; and one or more cannabis extracts disposed in the cosmetically or pharmaceutically acceptable carrier.

In one embodiment the composition includes potassium, zinc, calcium, and rubidium; potassium, zinc, and calcium; potassium, zinc, and rubidium; potassium, calcium, and rubidium; zinc, calcium, and rubidium; potassium, and zinc; potassium, and calcium; potassium and rubidium; zinc and calcium; zinc and rubidium; or calcium and rubidium. The composition may include potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition and within that range all variation and incremental variations are included. The composition comprises potassium, zinc, calcium, and rubidium. The composition comprises 0.01-5% Potassium, 0.01-5% Zinc, 0.01-1% Calcium, and 0.01-1% Rubidium. In one embodiment the composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

The present invention provides a method of regulating skin condition comprising applying to the skin of a subject in need thereof the topical composition comprising an effective amount of one or more selected from potassium, zinc, calcium, and rubidium and an effective amount of one or more cannabis extracts in a cosmetically acceptable vehicle. The skin condition to be treated is selected from the group consisting of eczema, seborrhea, psoriasis, xerosis, neoplastic growths, dermatitis, folliculitis, rosacea and acne. The composition comprises potassium, zinc, calcium, and rubidium. The composition comprises 0.01-5% Potassium, 0.01-5% Zinc, 0.01-1% Calcium, and 0.01-1% Rubidium. The composition comprises potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition. The composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexyiphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

The present invention provides a method for providing a benefit to human skin comprising: providing skin in need of treatment; and applying topically to skin a composition comprising an effective amount of one or more selected from potassium, zinc, calcium, and rubidium and an effective amount of one or more cannabis extracts in a cosmetically acceptable vehicle. The skin benefit is selected from the group consisting of: treatment of prevention of a sign of skin aging; treatment and/or prevention of fine lines or wrinkles; reduction of skin pore size; improvement in skin thickness, plumpness, and/or tautness; improvement in skin suppleness and/or softness; improvement in skin tone, radiance, and/or clarity; improvement in skin texture and/or promotion of retexturization; improvement in skin barrier repair and/or function; improvement in appearance of skin contours; restoration of skin luster and/or brightness; replenishment of essential nutrients and/or constituents in the skin; improvement of skin appearance decreased by menopause; improvement in skin moisturization and/or hydration; increase in and/or preventing loss of skin elasticity and/or resiliency; improvement in procollagen and/or collagen synthesis; treatment and/or prevention of skin sagging or atrophy; enhancing exfoliation and/or reducing dryness; treatment and/or prevention of skin hyper-pigmentation; treatment and/or prevention of inflammation; treatment and/or prevention of excess sebum output; and treatment and/or prevention of cellulite. The composition comprises potassium, zinc, calcium, and rubidium. The composition comprises 0.01-5% Potassium, 0.01-5% Zinc, 0.01-1% Calcium, and 0.01-1% Rubidium. The composition comprises potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition. The composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

The present invention provides a cosmetic composition comprising: a physiologically acceptable medium comprising one or more cannabis extracts and one or more selected from potassium, zinc, calcium, rubidium. The composition comprises potassium, zinc, calcium, and rubidium. The composition comprises 0.01-5% Potassium, 0.01-5% Zinc, 0.01-1% Calcium, and 0.01-1% Rubidium. The composition comprises potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition. The composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

The present invention provides a method for treating the cutaneous manifestations of aging and/or photo-aging, the method comprising: providing a composition comprising an effective quantity of a physiologically acceptable topical carrier comprising one or more cannabis extracts and one or more selected from potassium, zinc, calcium, rubidium. The composition comprises potassium, zinc, calcium, and rubidium. The composition comprises 0.01-5% Potassium, 0.01-5% Zinc, 0.01-1% Calcium, and 0.01-1% Rubidium. The composition comprises potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition. The composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

The present invention provides a topical pharmaceutical composition for application to a keratinous tissue comprising: a pharmaceutically acceptable carrier; one or more selected from potassium, zinc, calcium, rubidium disposed in the pharmaceutically acceptable carrier; and one or more peptides disposed in the pharmaceutically acceptable carrier. The composition comprises potassium, zinc, calcium, and rubidium. The composition comprises 0.01-5% Potassium, 0.01-5% Zinc, 0.01-1% Calcium, and 0.01-1% Rubidium. The composition comprises potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition. The composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

The present invention provides a topical pharmaceutical composition to aid in wound healing of a keratinous tissue comprising a pharmaceutically acceptable carrier; one or more selected from potassium, zinc, calcium, rubidium disposed in the pharmaceutically acceptable carrier; and one or more peptides disposed in the pharmaceutically acceptable carrier. The composition comprises potassium, zinc, calcium, and rubidium. The composition comprises 0.01-5% Potassium, 0.01-5% Zinc, 0.01-1% Calcium, and 0.01-1% Rubidium. The composition comprises potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition. The composition comprises potassium and zinc. The composition comprises potassium and zinc and calcium or rubidium. The cannabis extract is a cannabinoid, a terpene or a terpenoid compound. The cannabis extract is one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. The composition further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene. The composition further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose. The cannabis extract is from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose. The composition is a topical composition is in the form of a lotion, cream, essence ointment, gel, emulsion, gel, pack, cosmetic liquid, ointments, eye cream, day cream, night cream, pharmaceuticals, stick or combination thereof. The composition further comprises at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “active ingredient” refers to an ingredient having a therapeutic, cosmetic or cosmeceutical effect.

By the term “pharmaceutical active agent” we include any compound, including pharmaceutical acceptable derivatives such as a salt, solvate and pro-drug and any composition which may be used for the curative and/or prophylactic treatment of a medical condition of a human or animal. Preferably, the pharmaceutical active agent possesses antibacterial, antiviral and/or antifungal activity. More preferably, the pharmaceutical active agent comprises an antibacterial agent.

As used herein, the phrase “topical application” refers to an application on the skin, hair, ears, mucous membranes, rectal application, and nasal application, as well as dental or gum application within the oral cavity.

As used herein, “peptide” refers to any composition that includes two or more amino acids joined together by a peptide bond. Peptides may be about 2 to about 200 amino acids or more in length, and generally correspond to a fragment of a full-length protein, where the fragment does not include all the amino acids of the native full-length protein. In some embodiments, the peptide may be from at least about 3, at least about 4, at least about 5, at least about 6, at least about 8, at least about 10, at least about 15, or at least about 20 amino acids in length. In some embodiments, the peptide may be no more than about 200, no more than about 100, no more than about 50, no more than about 30, or no more than about 20 amino acids in length. For example, in some embodiments, the peptide includes less than about 20 amino acids, less than about 15 amino acids, less than about 10 amino acids, or about six amino acids. It further will be appreciated that peptides may contain amino acids other than the 20 amino acids commonly referred to as the 20 naturally-occurring amino acids, and that many amino acids, including the terminal amino acids, may be modified in a given peptide, either by natural processes such as glycosylation and other post-translational modifications, or by chemical modification techniques, such as those well known in the art. Among the known modifications which may be present in peptides of the present invention include, but are not limited to, acetylation, acylation, ADP-ribosylation, amidation, branching, cross-linking, cyclization, disulfide bond formation, demethylation, glycosylation, hydroxylation, iodination, methylation, oxidation, phosphorylation, prenylation, racemization, selenoylation, sulfation, and ubiquitination.

As used herein, “therapeutically effective amount,” “amount effective” or an “effective amount” to be an amount sufficient to effect treatment when administered to a subject in need of treatment. In the case of the embodiments of the present invention, a therapeutically effective amount can include, but is not limited to, an amount for increasing energy levels and/or alleviating fatigue in a patient

As used herein, “wrinkle” or “wrinkling” refers to both fine wrinkling and/or coarse wrinkling. Fine wrinkling or fine lines refers to superficial lines and wrinkles on the skin surface. Coarse wrinkling refers to deep furrows, particularly deep lines/wrinkles on the face and around the eyes, including expression lines such as frown lines and wrinkles, forehead lines and wrinkles, crow's feet lines and wrinkles, nasolabial folds, and marionette lines and wrinkles. Forehead lines and wrinkles refer to superficial lines and/or deep furrows on skin of the forehead. Crow's feet lines and wrinkles refer to superficial lines and/or deep furrows on skin around the eye area. Marionette lines and wrinkles refer to superficial lines and/or deep furrows on skin around the mouth.

As used herein “collagen” is used interchangeably with “collagen I” or “collagen type I,” the type present in skin as a dermal matrix component. Collagen I is composed of three protein chains wound together in a tight triple helix, which provides a tensile strength greater than that of steel. It is created by fibroblasts, specialized skin cells located in the dermis. Formation involves the production of preprocollagen I by ribosomes along the rough endoplasmic reticulum (RER); conversion to procollagen I and formation of the triple helical structure within the RER; and eventual formation of tropocollagen outside the cell, the form that aggregates to give collagen fibrils and then fibers. Collagen gives skin firmness, strength, durability, and a youthful smooth, plump appearance.

As used herein “Cannabis extract” includes a Cannabis oil. As used herein, a “Cannabis oil” is an extract formed by contacting at least a part of a Cannabis plant with an oil. The extracting oil may optionally be removed. Extracting oils may be selected from olive oil, hemp oil, sesame oil, coconut oil, vegetable oil, canola oil, grape seed oil, almond oil, medium-chain triglyceride (MCT) oil, and any other edible oil, or a combination thereof. As used herein “Cannabis extract” includes a diverse array of secondary metabolites, including cannabinoids, terpenes and terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract, and season.

As used herein “cannabinoid” as used herein relates to any cannabinoid that have been isolated from a Cannabis plant or synthetically created to have activity involving the endocannabinoid system.

As used herein “cannabinoid fraction” is used to describe the combination of cannabinoid compounds present in the Cannabis extract.

As used herein “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)ₙ, where n is the number of linked isoprene units. Terpenoids are terpene compounds that have been further metabolised in the plant, typically through an oxidative process, and therefore usually contain at least one oxygen atom.

The term “terpene fraction” is used to describe the combination of terpene and terpenoid compounds present in the Cannabis extract.

It will be understood by a person skilled in the relevant art that the compositions of the present invention can be formulated into pharmaceutical compositions for administration in a manner customary for administration of such materials using standard pharmaceutical formulation chemistries and methodologies, all of which are readily available to a person skilled in the relevant art. It will also be understood by a person skilled in the relevant art that such pharmaceutical compositions may include one or more excipients, carriers, stabilizers or other pharmaceutically inactive compounds, such as, but not limited to, wetting or emulsifying agents, pH buffering substances, hydroxypropylcellulose, starch, silicon dioxide, gelatin, magnesium stearate, microcrystalline cellulose and the like. Pharmaceutically acceptable salts can also be included therein. A thorough discussion of pharmaceutically acceptable excipients, vehicles and auxiliary substances is available in Remington's. Pharmaceutical Sciences (Mack Pub. Co. N.J. 1991). Such pharmaceutical compositions can be prepared as oral or transdermal preparations. The therapeutically effective doses may vary according to body weight and the timing and duration of administration will be determined by specific clinical research protocols.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

Extracts of Cannabis may be prepared by any means known in the art. The extracts may be formed from any part of the Cannabis plant containing cannabinoid, terpene and terpenoid compounds. Extracts may be formed by contacting an extractant with a leaf, seed, trichome, flower, keif, shake, bud, stem or a combination thereof. In some embodiments, the extract is formed from the flowers and shake of a Cannabis plant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a solvent (e.g. ethyl acetate, polyethylene glycol, etc.) or a supercritical fluid (e.g. liquid CO₂). The extractant may be completely or partially removed prior to incorporation of the Cannabis extract into the pharmaceutical composition, or it may be included in the pharmaceutical composition as a carrier. The extractant may be removed by heating the extract optionally under reduced pressure. It will be appreciated that some of the more volatile plant metabolites (such as terpenes) may also be removed with the extractant. Accordingly, in some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and pressure to the plant material. Typically, in these embodiments, no extractant is required.

For example, the Cannabis extract comprises a cannabinoid fraction and a terpene fraction. In some embodiments, the Cannabis extract contains high amounts (e.g. greater than 75% by weight) of the cannabinoid fraction. In some embodiments, the Cannabis extract may comprise the cannabinoid fraction in an amount of about 75% to about 99.999% by weight, for example, about 80% to about 99.999%, about 80% to about 99.99%, about 80% to about 99.9%, or about 80% to about 99.5% by weight of the Cannabis extract. In some embodiments, the Cannabis extract comprises about 0.001% to about 20% by weight of non-cannabinoids, for example, about 0.001% to about 15% by weight or about 0.001% to about 10% by weight non-cannabinoids.

In some embodiments, one or more additional compounds (e.g. cannabinoid, terpene or terpenoid compounds) may be added to the Cannabis extract. The addition of compounds may be to compensate for natural variations in the relative amounts of certain compounds being expressed in the Cannabis plant. The added compounds may be synthetic versions of the desired compounds, they may be purified compounds obtained from other Cannabis extracts, or they may be added by blending two or more extracts.

To date, over 100 cannabinoids have been identified in Cannabis plants. A comprehensive list of these cannabinoids may be found in Mahmoud A. El Sohly and Waseem Gul, “Constituents of Cannabis sativa.” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVAC3A); (.+−.)-Cannabichromene CBC-C5, (.+−.)-Cannabichromenic acid A CBCA-C5 A, (.+−.)-Cannabivarichromene, (.+−.)-Cannabichromevarin CBCV-C3, (.+−.)-Cannabichromevarinic acid A CBCVA-C3 A); (−)-Cannabidiol CBD-C5, Cannabidiol momomethyl ether CBDMC5, Cannabidiol-C4 CBD-C4, (−)-Cannabidivarin CBDVC3, Cannabidiorcol CBD-CI, Cannabidiolic acid CBDA-C5, Cannabidivarinic acid CBDVA-C3); Cannabinodiol CBNDC5, Cannabinodivarin CBND-C3); Δ9-Tetrahydrocannabinol C5, Δ9-Tetrahydrocannabinol-C4, Δ9-THCC4, Δ9-Tetrahydrocannabivarin, Δ9-THCV-C3, Δ9-Tetrahydrocannabiorcol, Δ9-Tetrahydrocannabinolic acid, Δ9-THCA-C5 A, Δ9-Tetrahydrocannabinolic acid B, Δ9-THCA-C5 B, Δ9-Tetrahydrocannabinolic acid-C4 A and/or B Δ9-THCA-C4 A and/or B, Δ9-Tetrahydro-cannabivarinic acid A, Δ9-THCVA-C3 A, Δ9-Tetrahydrocannabiorcolic acid A and/or B Δ9-THCOA-CI A and/or B), (−)-Δ8-trans-(6aR,10aR)-Δ8-Tetrahydrocannabinol (−)-Δ8-trans-(6aR,10aR)-Tetrahydrocannabinolic acid A .DELTA.sup.8-THCA-C5 A, (−)-(6a5,10aR)-Δ9-Tetrahydrocannabinol (−)-cis-Δ9-THC-C5); Cannabinol CBN-C5, Cannabinol-C4 CBN-C4, Cannabivarin CBN-C3, Cannabinol C2 CBN-C2, Cannabiorcol CBN-CI, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (.+−.)-(9R,10S/9S,10R)-); Cannabitriol (.+−.)-cis-CBT-C5, (−)-(9R,10R)-trans-10-O-Ethyl-cannabitriol (−)-trans-CBT-OEt-C5, (.+−.)-(9R,10R/9S,10S)-Cannabitriol-C3 (.+−.)-trans-CBT-C3, 8,9-Dihydroxy-Δ6a(10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-,6,.sup.9-tetrahydrocannabinol (−)-Cannabitetrol, 10-Oxo-Δ6a(10a)tetrahydrocannabinol OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5), (−)-Δ7-trans-(1R,3R,6R)-Isotetrahydrocannabinol, (.+−.)-Δ7-1,2-cis-(1R,3R,6S/1S,3S,6R)-Isotetrahydrocannabivarin, (−)-Δ7-trans-(1R,3R,6R)-Isotetrahydrocannabivarin; (.+−.)-(1aS,3aR,8bR,8cR)-Cannabicyclol CBL-C5, (.+−.)-(1aS,3aR,8bR,8cR)-Cannabicyclolic acid A CBLA-C5 A, (.+−.)-(1aS,3aR,8bR,8cR)-Cannabicyclovarin CBLV-C3; Cannabicitran CBTC5; Cannabichromanone CBCN-C5, CannabichromanoneC3 CBCN-C3, and Cannabicoumaronone CBCON-C5.

The Cannabis extract may comprise at least 75% by weight of a main cannabinoid. The main cannabinoid may be Δ9-tetrahydrocannabinol (THC) or cannabidiol (CBD). The Cannabis extract may comprise the main cannabinoid in an amount of at least 75%, 76%, 77%, 78%, 79%, 80%, 81%, 82%, 83%, 84%, 85%, 86%, 87%, 88%, 89%, 90%, 91%, 92%, 93%, 94%, or 95% by weight of the extract.

Typically, the Cannabis extract further comprises one or more secondary cannabinoids. The secondary cannabinoids may be selected from Cannabinodiol (CBN), Cannabichromanone (CBC), Δ9-Tetrahydrocannabinolic acid (THCA) and Cannabigerol (CBG). THC or CBD may also be present in the Cannabis extract as a secondary cannabinoid. Typically, each secondary cannabinoid is present in an amount from 0.001% to about 20% by weight of the extract, for example, about 0.001% to about 15% or about 0.01% to about 15% by weight of the extract.

In some embodiments, certain cannabinoids may be absent, or present in non-detectable amounts (e.g. less than 0.001% by weight of the analyte). In some embodiments, the Cannabis extract may exclude one or more of the following cannabinoids: Δ9-Tetrahydrocannabinolic acid (THCA), Cannabidiol (CBD), Δ9-Tetrahydrocannabivarin (THCV), Cannabidiolic acid (CBDA), Cannabigerolic acid (CBGA), Cannabinodiol (CBN) and Cannabichromanone (CBC).

The Cannabis extract comprises non-cannabinoid compounds, which typically includes a terpene fraction, i.e. terpenes and terpenoids. In some embodiments, the Cannabis extract comprises a terpene fraction in an amount of less than 20% by weight, for example, less than 19%, 18%, 17%, 16%, 15%, 10%, 9%, 8%, 7%, 6%, 5%, 4%, 3%, 2% or 1% by weight of the extract. In some embodiments, the Cannabis extract may comprise terpene and terpenoid compounds in an amount of more than 0.001% by weight of the extract, for example, more than 0.001%, 0.005%, 0.01%, 0.05%, 0.1%, 0.5%, or 1% of the total weight of the extract. In some embodiments, the Cannabis extract comprises about 0.001% to about 20% by weight of terpene and terpenoid compounds, for example, about 0.001% to about 15% by weight, about 0.001% to about 10% by weight, about 0.001% to about 6% by weight or about 0.001% to about 5% by weight of the composition.

Typically, the terpene fraction in the plant material used to form the extract may have a different terpene/terpenoid profile than the terpene profile of the final extract, both in terms of the amounts of specific compounds in the terpene fraction and the weight of the terpene fraction relative to the other components. For example, a Cannabis flower may comprise about 20% by weight cannabinoids and about 3% by weight terpenes. Following extraction and concentration (i.e. removal of the extractant), the amount of cannabinoids may increase to an amount of about 50-90% by weight and the terpene fraction may amount to about 0.1-6% by weight of the Cannabis extract. This typical scenario shows that while the cannabinoids are concentrated when the extractant is removed, the relative amount of the terpene fraction is reduced, likely due to the volatility of many of the terpenes/terpenoids present in the terpene fraction. Therefore, the profile of the terpene fraction present in the Cannabis extract is significantly different from the profile of the terpene fraction that exists in Nature.

A variety of terpenes and terpenoids have also been identified in Cannabis extracts, including monoterpenes, monoterpenoids, sesquiterpenes and sesquiterpenoids. For example, the following terpenes and terpenoids have been identified in Cannabis extracts: Alloaromadendrene, allyl hexanoate, benzaldehyde, (Z)-a-cis-bergamotene, (Z)-a-trans-bergamotene, -bisabolol, epi-a-bisabolol, -bisabolene, borneol (camphol), cis-y-bisabolene, bomeol acetate (bomyl acetate), .alpha.-cadinene, camphene, camphor, cis-carveol, caryophyllene (-caryophyllene), .alpha.-humulene (.alpha.-caryophyllene), .gamma.-cadinene, .DELTA.-3-carene, caryophyllene oxide, 1,8-cineole, citral A, citral B, cinnameldehyde, .alpha.-copaene (aglaiene), .gamma.-curcumene, -cymene, -elemene, .gamma.-elemene, ethyl decdienoate, ethyl maltol, ethyl propionate, ethylvanillin, eucalyptol, .alpha.-eudesmol, -eudesmol, .gamma.-eudesmol, eugenol, cis-famesene ((Z)-farnesene), trans-.alpha.-farnesene, trans-famesene, trans-.gamma.-bisabolene, fenchone, fenchol (norbomanol, -fenchol), geraniol, .alpha.-guaiene, guaiol, methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, -linolool), .alpha.-longipinene, menthol, .gamma.-muurolene, myrcene (-myrcene), nerolidol, trans-nerolidol, nerol, -ocimene (cis-ocimene), octyl acetate, .alpha.-phellandrene, phytol, .alpha.-pinene (2-pinene), -pinene, pulegone, sabinene, cis-sabinene hydrate (cis-thujanol), -selinene, .alpha.-selinene, .gamma.-terpinene, terpinolene (isoterpine), terpineol (.alpha.-terpineol), terpineol-4-ol, .alpha.-terpinene (terpilene), .alpha.-thujene (origanene), vanillin, viridiflorene (ledene), and .alpha.-ylange.

The terpene fraction may comprise one or more of beta-myrcene, linalool, nerolidol, limonene, alpha-bisabolol, camphene, delta-s-carene, beta-caryophyllene, caryophyllene oxide, p-cymene, geraniol, humulene, ocimene, pinene, and alpha-terpinene. Preferably, the extract comprises beta-myrcene. It is believed that beta-myrcene may enhance the bioavailability of the cannabinoids present in the extract. Beta-myrcene may be present in an amount of from 0% to about 40% by weight of the extract. In some embodiments, beta-myrcene is present in an amount of about 0-40% by weight of the terpene fraction, for example, from 0.001% to about 25%, 5.1% to 29% or about 5.5% to about 25% of the terpene fraction. The terpene fraction may further comprise one or more of linalool, nerolidol and limonene. When present, the limonene may be present in an amount of at least about 5.4% by weight of the terpene fraction, for example, from about 5.5% to about 50% or about 5.5% to about 20% by weight of the terpene fraction. Limonene is a cyclic monoterpene having the molecular formula C₁₀H₁₆. There are a number of different naturally occurring isomers; however, the most common form is the dextrorotatory isomer, namely D-limonene. Linalool is a terpenoid that is found in many flower and spice plants having the molecular formula C₁₀H₁₈. It is believed that when linalool is present in a Cannabis extract, that is may provide a sedative effect. In some embodiments, linalool may be present in an amount of at least 0.05% by weight of the terpene fraction. In some preferred embodiments, linalool is present in an amount of greater than 4.5% by weight (e.g. at least 5% by weight of the terpene fraction). In other embodiments, linalool is present in an amount of from 0.05% to 25% by weight of the terpene fraction, for example, from 0.1% to 20% or 5% to 20% by weight of the terpene fraction.

Nerolidol is a sesquiterpenoid having the molecular formula of C₁₅H₂₆O. It exists in Nature in two isomeric forms, namely nerolidol 1 and nerolidol 2, which differ in the geometry around a central olefin, i.e. either cis or trans isomers. The extract may comprise nerolidol (i.e. nerolidol 1 and nerolidol 2) in an amount of at least 0.001% by weight of the terpene fraction, for example, from 0.01% to 20% by weight of the terpene fraction. Nerolidol 2 may be present in a greater amount relative to nerolidol 1. In some embodiments, nerolidol 1 may be absent (or present in an amount below the limit of detection). In some embodiments, nerolidol 2 may be absent (or present in an amount below the limit of detection). In some embodiments, nerolidol 1 and nerolidol 2 are absent (or present in an amount below the limit of detection). Nerolidol 1 may be present in the extract in an amount of at least about 0.001% by weight of the terpene fraction, for example, from 0.001% to 20% or 0.001 to 15% by weight of the terpene fraction. Nerolidol 2 may be present in the extract in an amount of at least about 0.001% by weight of the terpene fraction, for example, from 0.001% to 30% or 1% to 25% by weight of the terpene fraction.

The Cannabis extract may also comprise a pinene (e.g. alpha-pinene and/or beta-pinene). Pinene is a bicyclic monoterpene having the molecular formula C₁₀H₁₆. Pinene is found in Nature in two isomeric forms: alpha-pinene and beta-pinene. The extract may comprise pinene (i.e. alpha-pinene and beta-pinene) in an amount of at least 5% by weight of the terpene fraction, for example, at least 6%, 7%, 8%, 9% or 10% by weight of the terpene fraction. Typically, alpha-pinene may be present in an amount greater than the amount of beta-pinene. The ratio of beta-pinene to alpha-pinene may be about 4:1. Alpha-pinene may be present in the extract in an amount of at least about 0.001% by weight of the terpene fraction, for example, from 0.001% to 30%, 0.001% to 20% or 5% to 20% by weight of the terpene fraction. Beta-pinene may be present in the extract in an amount of at least about 0.001% by weight of the terpene fraction, for example, 0.001% to 25%, 1% to 25% or 1% to 10% by weight of the terpene fraction.

The terpene fraction may also comprise beta-caryophyllene. Beta-caryophyllene may be present in an amount of at least 0.001% by weight of the terpene fraction, for example, from 0.001% to 20% or 0.001% to 10% of the terpene fraction.

The terpene fraction may also comprise caryophyllene oxide. Caryophyllene oxide may be present in an amount of at least 0.001% by weight of the terpene fraction, for example, from 0.001% to 50%, 5% to 40%, 10% to 40% or 20% to 40% by weight of the terpene fraction.

In some embodiments, the extract further comprises humulene. It is believed that that humulene may enhance the sedative properties of the extract. Humulene is also sometimes called alpha-caryophyllene.

The Cannabis extract may also include ocimene. Ocimene may be present in an amount of at least 0.001% by weight of the terpene fraction, for example, from 0.001% to 20% or 0.001% to 5% by weight of the terpene fraction.

In some embodiments, specific terpenes or terpenoids may be absent, or present in non-detectable amounts (e.g. less than 0.001% by weight of the analyte). In some embodiments, one or more of the following terpenes or terpenoids are absent, or present in non-detectable amounts: alpha-bisabolol, delta-s-carene, geraniol, guaiol, isopulegol, limonene, nerolidol 1, nerolidol 2, gamma-terpinene, and terpinolene.

Other liquid form preparations include those prepared by combining the Cannabis extract with one or more naturally derived oils (e.g. an essential oil) or waxes. An “essential oil” is an oil derived by extraction (e.g. steam extraction, or contacting the plant material with an extractant) or pressing, which contains primarily hydrophobic, and generally fragrant, components of the plant material. Suitable naturally derived oils and waxes include any of those mentioned for the topical delivery system described above, including: Bergamot essential oil, Cedarwood essential oil, Chamomile essential oil, Clary sage essential oil, Cypress essential oil, Eucalyptus essential oil, Fennel essential oil, Frankincense essential oil, Geranium essential oil, Hyssop essential oil, Jasmine essential oil, Juniper essential oil, Lavender essential oil, Lemon essential oil, Lemongrass essential oil, Marjoram essential oil, Melaleuca essential oil, Myrrh essential oil, Myrtle essential oil, Neem essential oil, Orange essential oil, Oregano essential oil, Palma rosa essential oil, Patchouli essential oil, Peppermint essential oil, Rose essential oil, Rosemary essential oil, Rosewood essential oil, Sage essential oil, Sandalwood essential oil, Tangerine essential oil, Tea tree essential oil, Thyme essential oil, Ylang ylang essential oil, Sesame oil, Olive oil, Arnica essential oil, Lavender Spike essential oil, Cinnamon Leaf essential oil, Rosemary Cineole essential oil, Coconut oil, Bees wax and Hemp oil.

For topical administration to the epidermis the active ingredients may be formulated as ointments, creams, oils or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The pharmaceutical compositions may be used to treat a skin disorder. As used herein, reference to “skin disorder” includes diseases and disorders of the skin. Diseases or disorders of the skin include: acne, alopecia areata, basal cell carcinoma, Bowen's disease, congenital erythropoietic porphyria, contact dermatitis, Darier's disease, dystrophic epidermolysis bullosa, eczema (atopic eczema), epidermolysis bullosa simplex, erythropoietic protoporphyria, fungal infections of nails, Hailey-Hailey disease, herpes simplex, hidradenitis suppurativa, hirsutism, hyperhidrosis, ichthyosis, impetigo, keloids, keratosis pilaris, lichen planus, lichen sclerosus, melanoma, melasma, pemphigus vulgaris, plantar warts (verrucas), pityriasis lichenoides, polymorphic light eruption, psoriasis, pyoderma gangrenosum, rosacea, scabies, shingles, squamous cell carcinoma, Sweet's syndrome, vitiligo, or a combination thereof.

The present invention provides a composition that includes potassium, zinc, calcium, rubidium or a combination thereof and one or more Cannabis extracts for use in wound care, e.g., chronic wounds. Chronic wounds are generally defined as wounds that have not healed after thirty days of consistent clinical treatment, and include diabetic ulcers, burns, pressure ulcers (bedsores), and venous stasis ulcers.

The present invention provides a composition that includes potassium, zinc, calcium, rubidium, or a combination thereof, and one or more Cannabis extracts for use in reducing the signs of aging and appearance of wrinkles. Facial wrinkling associated with aging is caused and exacerbated by numerous factors. Beyond the physiological pathways, molecular mechanisms involved in facial aging include changes in collagen conformation, elastin polypeptide degradation, and problems of the skin lipid matrix. The present invention provides a cosmeceutical composition that includes potassium, zinc, calcium, rubidium, or a combination thereof, and one or more Cannabis extracts, for use in reducing the signs of aging and appearance of wrinkles. The present invention is effective in controlling a group of proteases and other enzymes associated with the breakdown of collagen and tissue in the skin. The breakdown of collagen is a key factor in the aging of skin. Proteases have recently been identified as a contributing factor affecting aging skin by destroying the collagen in the dermis. Protease activity began at the cellular level in the dermis in the 21 to 40 age group. The activity became very high in the 41 to 60 age group resulting in a greatly reduced level of collagen. Recent studies also establish that changes can be significantly reduced by inhibiting soluble N-ethylmaleimide-sensitive factor attachment protein receptor (SNARE) complex formation, a core of membrane proteins that mediate neuronal exocytosis. Their inhibition by short synthetic peptides, used in the instant composition, can decrease facial wrinkle formation and aging. The overproduction and release of cate-cholamines and encouraged the formation of wrinkles and fine lines. Current compositions include a day cream, a night cream, an eye cream, plus a product for post Retin-A or laser repair usage, which is a PEG-based wound and burn formula containing the present invention. This composition is extremely beneficial when used with exfoliating products and laser repair, with studies showing greater than a 50% improvement in healing time.

The present invention provides a composition that includes potassium, zinc, calcium, rubidium, or a combination thereof, and one or more Cannabis extracts. One embodiment includes water and glycerin and one or more selected from cannabinoid, terpene or terpenoid compounds. The Cannabis extracts may include Cannabinodiol (CBN), Cannabichromanone (CBC), cannabidiol (CBD), Cannabigerol (CBG), Δ9-Tetrahydrocannabinolic acid (THCA) and Δ9-Tetrahydrocannabinol (THC). The Cannabis extract may further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene.

The composition of potassium, zinc, calcium, rubidium, or a combination thereof, and one or more Cannabis extracts reduce the degree of existing facial wrinkles and has been demonstrated effective against their development. Facial wrinkling associated with aging is caused and exacerbated by many factors. Beyond the physiological pathways, molecular mechanisms involved in facial aging include changes in collagen conformation, elastin polypeptide degradation, and problems of the skin's lipid matrix.

The present invention provides a composition that includes potassium, zinc, calcium, rubidium or a combination thereof and one or more Cannabis extracts. When it is applied to the skin, it significantly reduces wrinkles due to aging and other skin conditions. In addition to having a family of additional active ingredients for rapid as well as long-lasting effect. Each of the compositions incorporate active ingredients particular to that product's usage. The other active ingredients help prevent the degrading action of natural skin aging.

The present invention provides a composition that includes potassium, zinc, calcium, rubidium, or a combination thereof, and one or more Cannabis extracts; helps reduce the breakdown of collagen, laminin, elastin, and other components of the skin. The present invention provides a composition that includes potassium, zinc, calcium, rubidium, or a combination thereof, and one or more Cannabis extracts, and has found age-related changes in the activity of these enzymes, and can reduce skin degradation and loss of skin tone, enhance growth factors, regulate MMPs, and reduces inflammation.

In the day and night creams a proprietary peptide is used to provide a protective effect against photo-induced aging, and it significantly stimulates synthesis of collagen I, IV, VII, XVII and nidogen I proteins. While these proteins of the dermis and dermal-epidermal junction usually decrease with age, rejuvenates the dermal structure causing aging skin to behave like younger skin.

In an eye cream formulation the composition that includes potassium, zinc, calcium, rubidium, or a combination thereof, and one or more Cannabis extracts, are effective in diminishing darkness and puffiness around the eyes and general skin discoloration. Poor circulation and capillary fragility are primary factors that exacerbate the appearance of under-eye circles.

In a day cream formulation the composition that includes potassium, zinc, calcium, rubidium, or a combination thereof, and one or more Cannabis extracts, further includes sesame protein is a skin-tightening agent that visibly smooth the skin and is a very effective base for application of make-up. Sun-screen may also be added to the product.

In a night cream formulation the composition that includes potassium, zinc, calcium, rubidium or a combination thereof and one or more Cannabis extracts. This protects the cell's antioxidant pool and has biostimulating properties while shielding from the consequences of oxidative stress.

The present invention provides a composition that includes potassium, zinc, calcium, rubidium or a combination thereof and one or more Cannabis extracts and Retin-A. The Retin-A products have at least 4 different dosage levels, ranging from 0.075% to 0.5%. The low dose acts well for the stimulation of the cellular activity but does not produce a strong inflammatory reaction. This is a great product for a broad spectrum of users, from younger to older.

Another aspect of the instant invention relates to cosmetic use of compositions including potassium, zinc, calcium, rubidium or a combination thereof and one or more Cannabis extracts. The cosmetic compositions surprisingly act to increase one or more of KLKs activity, hyaluronic acid production, and collagen synthesis; and/or to decrease one or more of metallocollagenase activity, PPARs signaling, TNFα production, and melanin synthesis, and accordingly find use in exfoliating, anti-aging, anti-lipid, anti-inflammatory, and/or skin (or hair) lightening products.

In some embodiments, a method for providing at least one benefit to human skin is provided, where the method comprises topically applying to skin in need thereof at least one composition described herein in a cosmetically acceptable vehicle. The composition will comprise an effective amount of a composition including potassium, zinc, calcium, rubidium, or a combination thereof, and one or more Cannabis extracts.

The compositions of the invention can be applied to skin in need of treatment, such as skin which suffers from a deficiency or loss in any of the foregoing attributes or conditions, or which would otherwise benefit from the composition's exfoliating, anti-aging, anti-lipid, anti-inflammatory and/or skin lightening effects, e.g., as described herein. For example, the composition including potassium, zinc, calcium, rubidium, or a combination thereof, and one or more Cannabis extracts, can be provided in a cosmetically acceptable vehicle, topically applied to a desired area of skin, and allowed to remain on the area in an amount effective to treat and/or prevent an unwanted feature or condition of the skin, and/or to improve the aesthetic appearance of the skin.

In certain preferred embodiments, the cosmetic compositions described herein can be used to treat and/or prevent signs of skin aging or other skin damage. Signs of skin aging include any dermatological signs of aging, including signs caused by intrinsic aging, or caused by extrinsic factors. The compositions may be applied to skin already showing visible signs of aging, or likely to show such signs, e.g., due to age or sun exposure.

An early sign of skin aging involves the gradual development of facial wrinkles, whether fine surface lines or deeper creases and folds. While wrinkling and other signs of aging are intrinsic to skin, the process may be accelerated by external factors, such as excessive exposure to the sun and other damaging elements, overactive facial expression muscles, frequent use of tobacco products, poor nutrition, or certain skin disorders. Fine surface lines that progress to deeper creases, deepening facial wrinkles due to repeated skin folding, and deep folds that develop with maturity are visible changes associated with aging.

Treating signs of skin aging refers to eradicating, reducing, ameliorating, or reversing one or more of the unwanted features associated with skin aging, e.g., by reducing loss of skin firmness or plumpness to a perceptible extent. For example, compositions and methods of the instant invention may be used to reverse or treat signs of skin aging once manifested. Preventing signs of skin aging refers to affording skin a benefit that serves to avoid, delay, forestall, or minimize one or more unwanted features associated with aging, e.g., by slowing the loss of firmness or plumpness as the skin eventually ages. That is, the compositions and methods of the instant invention may be employed prophylactically, e.g., to forestall signs of skin aging in individuals that have not yet manifested signs of skin aging, most commonly in individuals under 25 years of age.

Compositions used to as anti-aging agents will comprise an effective amount of a composition including potassium, zinc, calcium, rubidium or a combination thereof and one or more Cannabis extracts to treat and/or prevent signs of aging. Some particularly preferred embodiments provide compositions for topical application which comprise an effective amount of composition including potassium, zinc, calcium, rubidium or a combination thereof and one or more Cannabis extracts to treat and/or prevent signs of aging. Treatment and/or prevention generally results in an improvement in one or more unwanted features and/or in the overall aesthetic appearance of the treated skin.

In certain embodiments, the cosmetic compositions described herein can be used to treat and/or prevent signs of skin aging or other skin damage. Signs of skin aging include any dermatological signs of aging, including signs caused by intrinsic aging, or caused by extrinsic factors. The compositions may be applied to skin already showing visible signs of aging, or likely to show such signs, e.g., due to age or sun exposure.

An early sign of skin aging involves the gradual development of facial wrinkles, whether fine surface lines or deeper creases and folds. While wrinkling and other signs of aging are intrinsic to skin, the process may be accelerated by external factors, such as excessive exposure to the sun and other damaging elements, overactive facial expression muscles, frequent use of tobacco products, poor nutrition, or certain skin disorders. Fine surface lines that progress to deeper creases, deepening facial wrinkles due to repeated skin folding, and deep folds that develop with maturity are visible changes associated with aging.

Treating signs of skin aging refers to eradicating, reducing, ameliorating, or reversing one or more of the unwanted features associated with skin aging, e.g., by reducing loss of skin firmness or plumpness to a perceptible extent. For example, compositions and methods of the instant invention may be used to reverse or treat signs of skin aging once manifested. Preventing signs of skin aging refers to affording skin a benefit that serves to avoid, delay, forestall, or minimize one or more unwanted features associated with aging, e.g., by slowing the loss of firmness or plumpness as the skin eventually ages. That is, the compositions and methods of the instant invention may be employed prophylactically, e.g., to forestall signs of skin aging in individuals that have not yet manifested signs of skin aging, most commonly in individuals under 25 years of age.

Compositions used as anti-aging agents will comprise an effective amount of a composition including potassium, zinc, calcium, rubidium, or a combination thereof, and one or more Cannabis extracts, to treat and/or prevent signs of aging. Some particularly preferred embodiments provide compositions for topical application which comprise an effective amount of composition including potassium, zinc, calcium, rubidium, or a combination thereof, and one or more Cannabis extracts to treat and/or prevent signs of aging. Treatment and/or prevention generally results in an improvement in one or more unwanted features and/or in the overall aesthetic appearance of the treated skin.

The improvement in the unwanted feature and/or overall aesthetic appearance can include one or more of the following: Reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; preventing, reducing, and/or diminishing the appearance and/or depth of lines and/or wrinkles; improving the appearance of suborbital lines and/or periorbital lines; reducing the appearance of crow's feet; rejuvenating and/or revitalizing skin, particularly aging skin; reducing skin fragility; preventing skin atrophy; improving skin tone, radiance, and/or clarity; preventing, reducing, and/or ameliorating skin sagging; improving skin firmness, plumpness, tautness, suppleness and/or softness; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause, such as essential nutrients or other skin constituents; ameliorating the effects of estrogen imbalance; improving communication among skin cells; increasing cell proliferation and/or multiplication; increasing skin cell metabolism decreased by aging and/or menopause; retarding cellular aging; improving skin moisturization and/or hydration; enhancing skin thickness; increasing skin elasticity and/or resiliency; improving procollagen and/or collagen synthesis; enhancing exfoliation; improving microcirculation; reducing dryness; and any combinations thereof.

In certain preferred embodiments, the compositions and methods of the invention are directed to the treatment and/or prevention of fine lines or wrinkles in the skin. In the case of treatment, the compositions are applied to skin in need of such treatment, by which is meant skin having wrinkles and/or fine lines. The fine lines and/or wrinkles may occur on any surface of the skin, including without limitation, the skin of the hands, arms, legs, neck, chest, and face, including the forehead. Preferably, the compositions are applied directly to the fine lines and/or wrinkles. For example, methods for treating fine lines and wrinkles may comprise topically applying a composition described herein to skin in need thereof, e.g., topically applying directly to a fine line and/or wrinkle in an amount and for a time sufficient to reduce the severity of the fine lines and/or wrinkles. The effect of a composition on the appearance of fine lines and wrinkles can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin).

In certain embodiments, the compositions and methods of the instant invention are directed to improving skin firmness, plumpness, and/or tautness. In certain embodiments, the compositions and methods of the instant invention are directed to increasing and/or preventing loss of skin elasticity. Elasticity of skin refers to the skin's springiness and/or resilience, due to the skin's ability to regain its original shape and size after deformation.

Loss of firmness, wrinkling and other signs of aging result in part from loss of skin collagen over time. As used herein, “increasing collagen synthesis” and related expressions refer to stimulating, inducing, or up-regulating procollagen and/or collagen production to increase the collagen content in an area of skin, preferably improving skin firmness and/or plumpness to a perceptible extent. For example, in some embodiments, collagen synthesis is increased by at least about 10%, at least about 25%, at least about 50%, at least about 75%, or at least about 90%, compared to the synthesis of collagen in the absence of the composition. The extent of collagen and/or collagen synthesis in the skin can be determined by appropriate assays, e.g., in vitro assays described herein or known in the art.

In certain embodiments, the compositions of the instant invention including potassium, zinc, calcium, rubidium, or a combination thereof, and one or more Cannabis extracts in an amount sufficient to decrease metalloproteinase activity in given area of skin when topically applied thereto.

Loss of hyaluronic acid over time also plays a role in skin aging. Hyaluronic acid is a glycosaminoglycan (GAG) found in the skin as part of the ECM. GAGs are long unbranched polymers of repeating disaccharide units, mainly composed of hexosamine, hexose, hexuronic acid moieties, or sulfates thereof. GAGs bind to proteins in the skin to form proteoglycans, which contribute to the growth, preservation, and repair of skin. Hyaluronic acid, in particular, has been reported to be responsible for hydration of the skin, nutrient exchange, and protection against free radicals.

In certain preferred embodiments, the compositions and methods of the instant invention are directed to improving skin moisturization and/or hydration. For example, in certain embodiments, the compositions of the instant invention including potassium, zinc, calcium, rubidium, or a combination thereof, and one or more peptides in an amount sufficient to increase hyaluronic acid production in a given area of skin when topically applied thereto. As used herein, “increasing hyaluronic acid production” and related expressions refer to stimulating, inducing, or up-regulating hyaluronic acid synthesis to increase the hyaluronic acid content in an area of skin, preferably improving skin hydration and/or resiliency by a perceptible amount. For example, in some embodiments, hyaluronic acid production is increased by at least about 10%, at least about 25%, at least about 50%, at least about 75%, or at least about 90%, compared to the production of hyaluronic acid in the absence of the composition. The extent of hyaluronic acid and/or hyaluronic acid production in the skin can be determined by appropriate assays, e.g., in vitro assays described herein or known in the art.

In some embodiments, a composition described herein is topically applied to the skin of the elbows, knees, ankles, feet, soles of the feet, heels, and the like, areas. In some preferred embodiments, a composition described herein is topically applied an area of dry skin.

In some embodiments, the cosmetic compositions for treating and/or preventing signs of skin aging can further comprise additional exfoliating and/or anti-aging agents. For example, the cosmetic composition comprising potassium, zinc, calcium, rubidium or a combination thereof and one or more Cannabis extracts in an amount effective to treat and/or prevent signs of skin aging. It is contemplated that synergistic improvements may be obtained with such combinations, in some embodiments.

Exemplary exfoliating agents include, without limitation, alpha-hydroxyacids, beta-hydroxyacids, oxaacids, oxadiacids, and their derivatives such as esters, anhydrides, and salts thereof. Suitable hydroxy acids include, for example, glycolic acid, lactic acid, malic acid, tartaric acid, citric acid, 2-hydroxyalkanoic acid, mandelic acid, salicylic acid, and derivatives thereof, as well as fruit enzymes, such as pineapple enzyme. A preferred additional exfoliating agent is glycolic acid.

Exemplary anti-aging agents include, without limitation, botanicals (e.g., Butea Frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; antioxidants, exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; anti-aging botanicals, keratolytic agents, desquamating agents, keratinocyte proliferation enhancers, and skin plumpers that serve as additional collagen enhancers to the skin, to name a few. An example of a suitable skin plumper is palmitoyl oligopeptide. Other skin plumping agents include other collagen and/or other glycosaminoglycan (GAG) enhancing agents. Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof. In some embodiments, the invention relates to synergistic action of one or more compositions described herein with TDPA, e.g., to provide enhanced anti-aging benefits to skin. Additional examples of anti-aging agents are provided below.

The compositions described herein can be formulated as a variety of skin care products for topical application. The composition may be formulated in a variety of product forms suitable for application to the skin (including the scalp and/or hair), such as, for example, a lotion, cream, serum, spray, aerosol, cake, ointment, essence, gel, paste, patch, pad, pencil, pomade, solution, towelette, mask, stick, foam, elixir, mousse, powder, bath salt, foaming cleanser, concentrate, or any other liquid, semi-solid, or solid form. Preferably the composition is formulated as a lotion, cream, essence, ointment, or gel, and in particular a lotion, toner pad or cleanser, e.g., for anti-acne products.

For example, the potassium, zinc, calcium, rubidium or a combination thereof may be present in an amount from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition.

The compositions can include a cosmetically acceptable vehicle. A cosmetically acceptable vehicle refers to any vehicle, for a cosmetic, drug or medicament that is suitable for use in direct, safe contact with human tissues and/or human hair, and may include, e.g., any diluent, solvent, carrier, filler, or the like. Such vehicles may take the form of any known in the art suitable for application to skin (or hair) and may include water (e.g., deionized water); vegetable oils; mineral oils; esters such as octal palmitate, isopropyl myristate, and isopropyl palmitate; ethers such as dicapryl ether and dimethyl isosorbide; alcohols such as ethanol and isopropanol; fatty alcohols such as cetyl alcohol, cetearyl alcohol, stearyl alcohol, and biphenyl alcohol; isoparaffins such as isooctane, isododecane, and isohexadecane; silicone oils such as cyclomethicone, dimethicone, dimethicone cross-polymer, polysiloxanes, and their derivatives, preferably organo-modified derivatives; hydrocarbon oils such as mineral oil, petrolatum, isoeicosane, and polyisobutene; polyols such as propylene glycol, glycerin, butylene glycol, pentylene glycol, and hexylene glycol; waxes such as beeswax and botanical waxes; or any combinations or mixtures of the foregoing. Based on the teachings herein, a person skilled in the art will be able to select a suitable vehicle, and/or in an amount thereof, such that one or more of the desired properties of the cosmetic compositions of the instant invention can be preserved. The vehicle may comprise an aqueous phase, an oil phase, an alcohol phase, a silicone phase, or mixtures thereof. The cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions, or the like having the appearance of a cream, gel, or microemulsions. The emulsion may include an emulsifier, such as a nonionic, anionic or amphoteric surfactant. The oil phase of the emulsion preferably has one or more organic compounds, including emollients; humectants, such as butylene glycol, propylene glycol, Methyl gluceth-20, and glycerin; other water-dispersible or water-soluble components, including thickeners such as veegum or hydroxyalkyl cellulose; gelling agents, such as high MW polyacrylic acid and mixtures thereof. The emulsion may have one or more emulsifiers capable of emulsifying the various components present in the composition. The compounds suitable for use in the oil phase include without limitation, vegetable oils; esters such as octyl palmitate, isopropyl myristate, and isopropyl palmitate; ethers such as dicapryl ether; fatty alcohols such as cetyl alcohol, stearyl alcohol, and behenyl alcohol; isoparaffins such as isooctane, isododecane, and isohexadecane; silicone oils such as dimethicones, cyclic silicones, and polysiloxanes; hydrocarbon oils such as mineral oil, petrolatum, isoeicosane, and polyisobutene; natural or synthetic waxes; and the like. The oil-containing phase may be composed of a single oil or mixtures of different oils. Suitable hydrophobic hydrocarbon oils may be saturated or unsaturated, have an aliphatic character, be straight or branched chained, or contain alicyclic or aromatic rings. Hydrocarbon oils include those having 6-20 carbon atoms, more preferably 10-16 carbon atoms. Representative hydrocarbons include decane, dodecane, tetradecane, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the trade name Permethyl 99 ATM are also contemplated to be suitable. Various commercially available C₁₆ isoparaffins, such as isohexadecane are also suitable. Examples of preferred volatile hydrocarbons include polydecanes, such as isododecane and isodecane, including for example, Permethyl-99A and the C₇-C₈ through C₁₂-C₁₅ isoparaffins.

The oil phase may comprise one or more waxes, including for example, rice bran wax, carnauba wax, ouricurry wax, candelilla wax, montan waxes, sugar cane waxes, ozokerite, polyethylene waxes, Fischer-Tropsch waxes, beeswax, microcrystalline wax, silicone waxes, fluorinated waxes, and any combination thereof. Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. Some preferred emulsifiers include soya sterol, cetyl alcohol, stearic acid, emulsifying wax, acrylates, silicone containing emulsifiers, and mixtures thereof. Other specific emulsifiers that can be used with the compositions described herein include, but are not limited to, one or more of the following: C.sub.10-30 alkyl acrylate cross polymer; Dimethicone PEG-7 isostearate; acrylamide copolymer; mineral oil; sorbitan esters; polyglyceryl-3-diisostearate; sorbitan monostearate; sorbitan tristearate; sorbitan sesquioleate; sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11th Edition 2006, the disclosure of which is hereby incorporated by reference. These emulsifiers typically will be present in the composition in an amount from about 0.001% to about 10% by weight, in particular in an amount from about 0.01% to about 5% by weight, and more preferably, from about 0.1% to about 3% by weight. Based on the teachings herein, a person skilled in the art will be able to select a suitable emulsifier, or any other materials described herein, and/or in an amount thereof, such that one or more of the desired properties of the cosmetic compositions of the instant invention can be preserved.

The oil phase may comprise one or more volatile and/or non-volatile silicone oils. Volatile silicones include cyclic and linear volatile dimethylsiloxane silicones. In some embodiments, the volatile silicones may include cyclodimethicones, including tetramer (D4), pentamer (D5), and hexamer (D6) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Non-volatile silicone oils will typically comprise polyalkylsiloxanes, polyarylsiloxanes, polyalkylarylsiloxanes, or mixtures thereof. Polydimethylsiloxanes are preferred non-volatile silicone oils. The non-volatile silicone oils will typically have a viscosity from about 10 to about 60,000 centistokes at 25° C., preferably between about 10 and about 10,000 centistokes, and more preferred still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted will various functional groups such as alkyl, aryl, amine groups, vinyl, hydroxyl, haloalkyl groups, alkylaryl groups, and acrylate groups, to name a few. The aqueous phase of the emulsion may include one or more additional solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like. The oil-containing phase will typically comprise from about 10% to about 99%, preferably from about 20% to about 85%, and more preferably from about 30% to about 70% by weight, based on the total weight of the emulsion, and the aqueous phase will typically comprise from about 1% to about 90%, preferably from about 5% to about 70%, and more preferably from about 20% to about 60% by weight of the total emulsion. The aqueous phase will typically comprise from about 25% to about 100%, more typically from about 50% to about 95% by weight water.

The compositions may include liposomes. The liposomes may comprise other additives or substances and/or may be modified to more specifically reach or remain at a site following administration. Additional suitable delivery vehicles include, e.g., niosomes, submicron emulsions, polymeric encapsulants, gels, creams, lotions, and combinations thereof.

The composition may optionally comprise other cosmetic actives and excipients, obvious to those skilled in the art including, but not limited to, fillers, emulsifying agents, surfactants, film formers, chelating agents, such as EDTA, gelling agents, thickeners, emollients, humectants, moisturizers, vitamins, minerals, viscosity and/or rheology modifiers, sunscreens, retinoids, hormonal compounds, alpha-hydroxy acids, alpha-keto acids, anti-mycobacterial agents, antifungal agents, antimicrobials, antivirals, analgesics, lipidic compounds, antineoplastics, immune system boosting agents, anti-allergenic agents, H1 or H2 antihistamines, anesthetics, antiseptics, insect repellents, skin cooling compounds, skin protectants, skin penetration enhancers, exfoliants, lubricants, fragrances, colorants, hypopigmenting agents, preservatives, stabilizers, pharmaceutical agents, photostabilizing agents, neutralizers, and mixtures thereof. In addition to the foregoing, the cosmetic compositions of the invention may contain any other compound for the treatment of skin conditions or disorders. Based on the teachings herein, a person skilled in the art will be able to select any of these or other materials, and/or in an amount thereof, such that one or more of the desired properties of the cosmetic compositions of the instant invention can be preserved. Preservatives may include, for example, alcohols, glycols, parabens, quaternary nitrogen-containing compounds, isothiazolinones, aldehyde-releasing agents, antioxidants, halogenated compounds, and combinations thereof. Illustrative alcohols include, e.g., phenoxyethanol, isopropyl alcohol, and benzyl alcohol; illustrative glycols include, e.g., propylene, butylene, and pentylene glycol; illustrative parabens include, e.g., methyl, propyl and butyl-parabens; illustrative quaternary nitrogen-containing compounds include, e.g., benzalkonium chloride and Quaternium 15; illustrative isothiazolinones include, e.g., methylisothiazolinone and methylchloroisothiazolinone; illustrative aldehyde-releasing agents include, e.g., DMDM hydantoin, imidazolidinyl urea, and diazolidinyl urea; illustrative antioxidants include, e.g., butylated hydroxytoluen and tocopherol, and illustrative halogenated compounds include, e.g., triclosan and chlorohexidine digluconate.

Various fillers and additional components may be added. Fillers are normally present in an amount of about 0 weight % to about 20 weight %, based on the total weight of the composition, preferably about 0.1 weight % to about 10 weight %. Suitable fillers include without limitation silica, treated silica, talc, zinc stearate, mica, kaolin, Nylon powders, polyethylene powder, starch, boron nitride, copolymer microspheres, and silicone resin microbeads and the like.

The number of applications of a composition to the skin that will generally be effective for producing one or more desired effects, as are described herein, and the period of time during which such applications are made, may vary widely, depending upon a variety of factors, such as the concentration of the one or more active agents that are present in the composition, the amount of the composition that is applied to the skin, the condition of the skin, the amount of the sun exposure and the type, age, sex, genetic predisposition and general health of the mammal, and may readily be determined by those having ordinary skill in the art using the information provided herein. While the skin may exhibit some improvement after only one application of a composition thereto, in order to obtain a more pronounced or full effect or benefit, it is typically beneficial to provide two or more applications to the skin, and more typically beneficial to provide at least about three applications of the composition to the skin (three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty and so forth applications) continuously over a period of at least about one day or week, or a series of days or weeks (one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty and so forth days or one, two, three, four, five, six, seven, eight, nine, ten, eleven, twelve, and so forth weeks). The compositions may, for example, be applied as a solid stick (or other solid form), a cream, a gel, a lotion, an ointment or other convenient form as frequently as once per 15 minutes (or per fewer minutes, such as 1, 5 or 10 minutes) and as infrequently as once per day or so or on an “as needed” basis (i.e., applied as believed to be necessary or desirable, or required, typically depending upon the types and severity of symptoms). Thus, as only by way of some examples, the following quantities of compositions within the present invention, or others, may be administered to a user per application (in g or ml), as well as any quantities in between: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9. 7.0, 7.1, 7.2, 7.3, 7.4, 7.5, 7.6, 7.7, 7.8, 7.9, 8.0. 8.1, 8.2, 8.3, 8.4, 8.5, 8.6, 8.7, 8.8, 8.9, 9.0. 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21, 22, 23, 24, 25, 26, 27, 28, 29, 30, 31, 32, 33, 34, 35, 36, 37, 38, 39, 40 and so forth.

In one aspect, the present invention provides a composition for topical application to the skin for repairing, improving or fully healing a skin disorder, disease or condition, or for causing the skin to experience a reduction in pain, soreness or itchiness, or an increase in soothing, softening or conditioning, or a combination thereof, comprising a cosmetically or pharmaceutically acceptable carrier; one or more selected from potassium, zinc, calcium, rubidium disposed in the cosmetically or pharmaceutically acceptable carrier; and one or more Cannabis extracts disposed in the cosmetically or pharmaceutically acceptable carrier in a combined amount that is effective for repairing, improving or healing the skin, or causing the skin to experience a reduction in pain, soreness or itchiness, or an increase in soothing, softening or conditioning, or a combination thereof, after a topical application of the composition to the skin; and (b) a base composition, wherein the base composition is present in the composition in an amount that is effective for permitting the base composition to function effectively as a carrier vehicle for the active agents when topically applied to the mammal's skin.

In still another aspect, the present invention provides a method for repairing, improving or fully healing a skin disorder, disease or condition of a mammal, or for causing the skin to experience a reduction in pain, soreness or itchiness, or an increase in soothing, softening or conditioning, or a combination thereof, comprising topically applying to the mammal's skin on a regular basis at least one or two applications of one of the above compositions, wherein the amount of the composition that is applied to the skin is effective for repairing, improving or fully healing a skin disorder, disease or condition of the mammal, or for causing the mammal's skin to experience a reduction in pain, soreness or itchiness, or an increase in soothing, softening or conditioning, or a combination thereof, and wherein the temperature of the composition is optionally elevated to a temperature above ambient temperature prior to topically applying the composition to the mammal's skin.

In still another aspect, the present invention provides a method for repairing, improving or fully healing a skin disorder, disease or condition of a mammal, or for causing the mammal's skin to experience a reduction in pain, soreness or itchiness, or an increase in soothing, softening or conditioning, or a combination thereof, consisting of (or consisting essentially of) topically applying to the mammal's skin on a regular basis at least one or two applications of one of the above compositions, wherein the amount of the composition that is applied to the skin of the mammal is an amount that is effective for repairing, improving or fully healing a skin disorder, disease or condition of the mammal, or for causing the mammal's skin to experience a reduction in pain, soreness or itchiness, or an increase in soothing, softening or conditioning, or a combination thereof.

In a preferred embodiment, the skin condition to be treated is one or more of the conditions that may be referred to collectively as the signs of skin aging. The appearance of skin normally changes with age, due to a number of internal factors associated with time. However, the skin can also be prematurely aged by virtue of its overexposure to environmental factors such as sun, pollution, or cigarette smoke. As used herein, the regulation of skin conditions resulting from aging is intended to encompass both the signs of chronoaging as well as photo- or environmentally-induced aging. The manifestations of the aging process are many, and may be both external (i.e., immediately visible) or internal (i.e., not immediately visible to the naked eye). Those skilled in the art will readily recognize the numerous examples of the signs of aging. Such examples include, without limitation, fine lines and wrinkles, deep wrinkles, pitting and bumps, increased pore size, keratosis, skin flakiness or roughness, unevenness or blotching of skin tone, yellowing of the skin, dark under eye shadows or circles, loss of skin elasticity, sagging (including puffiness in the eye area and jowls), elastosis, loss of skin firmness or tightness, hyperpigmentation, age spots and freckles, abnormal differentiation, hyperkeratinization, collagen breakdown, spider veins, or telangiectasia, among others.

The composition can further contain a surface-active agent. Surface-active agents (also termed “surfactants”) include any agent linking oil and water in the composition, in the form of emulsion. The composition contains a non-ionic surfactant. Nonlimiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, brij 38, brij 52, brij 56 and brij Wi, a sucrose ester, a partial ester of sorbitol and its anhydrides, sorbitan monolaurate, sorbitan monolaurate a monoglyceride, a diglyceride, isoceteth-20 and mono-, di- and tri-esters of sucrose with fatty acids.

In an embodiment, a composition includes one or more additional components. Such additional components include but are not limited to buffering agents, bulking agents, chelating agents, cleansers, colorants, conditioners, diluents, dyes, emollients, fragrances, humectants, pearlescent aids, perfuming agents, permeation enhancers, pH-adjusting agents, preservatives, protectants, skin penetration enhancers, softeners, solubilizers, sunscreens, sun blocking agents, sunless tanning agents, and viscosity modifiers. As is known to one skilled in the art, in some instances a specific additional component may have more than one activity, function or effect. In an embodiment, the additional component is a pH adjusting agent or a buffering agent. Suitable buffering agents include but are not limited to acetic acid, adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof. In an embodiment, the additional component is an emollient. Suitable emollients include but are not limited to mineral oil, lanolin oil, coconut oil, cocoa butter, olive oil, aloe vera extract, jojoba oil, castor oil, fatty acids, fatty alcohols, diisopropyl adipate, hydroxybenzoate esters, benzoic acid esters of C₉₋₁₅ alcohols, isononyl iso-nonanoate, silicone oils, polyethers, C₁₂₋₁₅ alkyl benzoates, oleic acid, stearic fatty acid, cetyl alcohols, hexadecyl alcohol, dimethyl polysiloxane, polyoxypropylene cetyl ether, polyoxypropylene butyl ether, and derivatives, esters, salts and mixtures thereof. In an embodiment, the additional component is a preservative. Suitable preservatives include but are not limited to alkyl benzoates, alkyl p-hydroxybenzoates, aloe vera extract, ascorbic acid, benzalkonium chloride, benzoic acid, benzoic acid esters of C₉₋₁₅ alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, potassium sorbate, silicone oils, sodium propionate, sodium benzoate, sodium bisulfite, sorbic acid, stearic fatty acid, vitamin E, vitamin E acetate and derivatives, esters, salts and mixtures thereof.

In an embodiment, the additional component is a skin penetration enhancer. Suitable skin penetration enhancers include but are not limited to acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl)ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacryl-ate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran, tetrahydrofurfural alcohol, transcutol, triethanolamine dodecyl benzene sulphonate, triethanolamine oleate, urea, water and derivatives, esters, salts and mixtures thereof.

Lubricants may be introduced into any of the formulations to reduce friction during application. This may be useful for producing a good skin feeling or ease of body cavity use. Any pharmaceutically or cosmetically acceptable lubricant may be used. Non limiting examples of general categories of lubricants are; polymeric substances such as celluloses, natrosol; hyaluronic acid; glycerin; silicones (e.g. dimethicone); and oil based lubricants such as plant based oils (e.g. olive oil, sweet almond oil; avocado oil and the like); petrolatum; and fats. In some situations it may be appropriate to select a polymeric agent having a viscosity and also a lubricating effect such that it has some adherence to the site of application but displays a reduced friction and is easier and more pleasant to use.

In addition the present invention may include an elemental metal, selected from the group consisting of silver, copper, zinc, mercury, tin, lead, bismutin, cadmium, chromium and/or gold.

The topical carrier in liquid hair or skin compositions may be water, common organic solvents, or mixtures thereof. Suitable common organic solvents are C₂-C₃ lower monohydric or polyhydric alcohols such as ethanol, propanol, isopropanol, glycerin, dimethylformamide, dimethylacetamide, and dimethylsulfoxide. In addition the present invention may include one or more selected from detersive surfactants, anionic surfactants, nonionic surfactants, amphoteric surfactants, zwitterionic surfactants, cationic surfactants, volatile carriers, azoles, selenium sulfides, sulfur, suspending agents, thickening agents, polymers, spreading agents, hydrocarbon oils, polyolefin, fatty esters, polyalkylene glycols.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

What is claimed is:

1. A topical composition for application to a keratinous tissue comprising:
a cosmetically or pharmaceutically acceptable carrier;
one or more selected from potassium, zinc, calcium, rubidium disposed in the cosmetically or pharmaceutically acceptable carrier; and
one or more cannabis extracts disposed in the cosmetically or pharmaceutically acceptable carrier.

2. The composition of claim 1, comprising potassium, zinc, calcium, and rubidium; potassium, zinc, and calcium; potassium, zinc, and rubidium; potassium, calcium, and rubidium; zinc, calcium, and rubidium; potassium, and zinc; potassium, and calcium; potassium and rubidium; zinc and calcium; zinc and rubidium; or calcium and rubidium.

3. The composition of claim 1, comprising 0.01-5% Potassium, 0.01-5% Zinc, 0.01-1% Calcium, and 0.01-1% Rubidium.

4. The composition of claim 1, comprising potassium, zinc, calcium, and rubidium in an individual concentration of from about 0.001 weight % to about 5 weight % based on the total weight of the composition; from about 0.01 weight % to about 3 weight % based on the total weight of the composition; and from about 0.1 weight % to about 2 weight %, or about 1 weight %, based on the total weight of the composition.

5. The composition of claim 1, comprising potassium and zinc; or potassium and zinc and calcium or rubidium.

6. The composition of claim 1, wherein the cannabis extract is a cannabinoid, a terpene or a terpenoid compound.

7. The composition of claim 1, wherein the cannabis extract comprises one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof.

8. The composition of claim 1, wherein the cannabis extract further comprises one or more secondary cannabinoids, beta-myrcene, linalool, nerolidol, alpha-bisabolol, camphene, delta-s-carene, beta-caryophyllene, caryophyllene oxide, p-cymene, geraniol, humulene, ocimene, pinene, and alpha-terpinene.

9. The composition of claim 1, further comprising one or more vitamins selected from Vitamin B9 (Folate) from about 25 mcg to about 2000 mcg per dose; and Vitamin B12 (Cobalamin) from about 250 mcg to about 2000 mcg per dose and more preferably Vitamin B9 (Folate) from about 250 mcg to about 400 mcg per dose; and Vitamin B12 (Cobalamin) from about 450 mcg to about 550 mcg per dose.

10. The composition of claim 1, wherein the cannabis extract comprises from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose.

11. The composition of claim 1, wherein the topical composition is in the form of a lotion, cream, essence ointment, gel, emulsion, gel, pack, cosmetic liquid, ointments, eye cream, day cream, night cream, pharmaceuticals, stick or combination thereof.

12. The composition of claim 1 further comprising at least one skin benefit agent selected from the group consisting of astringents, antioxidants, free radical scavengers, anti-acne agents, antimicrobial agents, antifungal agents, chelating agents, anti-aging agents, anti-wrinkle agents, analgesics, skin lightening agents, skin conditioning agents, anti-irritants, anti-inflammatories, anti-cellulite agents, humectants, emollients, organic sunscreens, inorganic sun protecting agents, chemical exfoliating agents, physical exfoliating agent, self-tanning agents, biologically active peptides; and mixtures thereof.

13. A method for providing a benefit to human skin comprising:
providing skin in need of treatment; and
applying topically to skin a composition comprising an effective amount of one or more selected from potassium, zinc, calcium, and rubidium and an effective amount of one or more cannabis extracts in a cosmetically acceptable vehicle.

14. The method according to claim 13, wherein the skin benefit is selected from the group consisting of: treatment of prevention of a sign of skin aging; treatment and/or prevention of fine lines or wrinkles; reduction of skin pore size; improvement in skin thickness, plumpness, and/or tautness; improvement in skin suppleness and/or softness; improvement in skin tone, radiance, and/or clarity; improvement in skin texture and/or promotion of retexturization; improvement in skin barrier repair and/or function; improvement in appearance of skin contours; restoration of skin luster and/or brightness; replenishment of essential nutrients and/or constituents in the skin; improvement of skin appearance decreased by menopause; improvement in skin moisturization and/or hydration; increase in and/or preventing loss of skin elasticity and/or resiliency; improvement in procollagen and/or collagen synthesis; treatment and/or prevention of skin sagging or atrophy; enhancing exfoliation and/or reducing dryness; treatment and/or prevention of skin hyper-pigmentation; treatment and/or prevention of inflammation; treatment and/or prevention of excess sebum output; and treatment and/or prevention of cellulite.

15. The method of claim 13, wherein the skin suffers from eczema, seborrhea, psoriasis, xerosis, neoplastic growths, dermatitis, folliculitis, rosacea or acne.

16. The method of claim 13, comprising potassium, zinc, calcium, and rubidium.

17. The method of claim 13, wherein the cannabis extract comprises one or more selected from cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, Δ9-tetrahydrocannabinol (THC or dronabinol), Δ8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof.

18. The method of claim 13, wherein the cannabis extract comprises from about 0.5 mg to about 1000 mg of cannabinoid per dose; about 25 mg to about 1000 mg of cannabinoid per dose; about 10 mg to about 500 mg of cannabinoid per dose; about 100 mg to about 200 mg of cannabinoid per dose; about 1 mg to about 30 mg of cannabinoid per dose; or about 2.5 mg to about 10 mg of cannabinoid per dose.
